(19) United States
(12) Patent Application Publication
(10) Pub. No.: US 2015/0165207 A1
(43) Pub. Date: Jun. 18, 2015

(54) METHOD AND DEVICE FOR RESPIRATORY AND CARDIORESPIRATORY SUPPORT

(71) Applicant: MEDISCI L.L.C., Fridley, MN (US)

(72) Inventor: Mustafa Karamanoglu, Fridley, MN (US)

(21) Appl. No.: 14/411,947

(22) PCT Filed: Jul. 1, 2013
(86) PCT No.: PCT/US2013/048893

§ 371 Date: Dec. 30, 2014

Related U.S. Application Data
(60) Provisional application No. 61/667,000, filed on Jul. 2, 2012.
(60) Provisional application No. 61/675,433, filed on Jul. 25, 2012.

(51) Int. Cl. A61N 1/36; A61M 5/14; A61B 5/15; A61B 5/00; A61B 5/0205; A61B 5/042; A61B 5/0215; A61M 25/09; A61B 17/12

(52) U.S. Cl. CPC A61N 1/36139; A61M 25/09; A61M 5/1408; A61B 5/150992; A61B 17/12109; A61B 5/02055; A61B 5/0422; A61B 5/02158; A61B 17/12136; A61B 5/4836; A61N 1/3611; A61N 1/36114; A61B 5/01

(57) ABSTRACT

A system and method for reducing a patient's exposure to mechanical ventilation delivers a series of nerve stimulation therapy regimes after determining whether a cardiac signal can be sensed by a most distal cardiac signal sensor along a lead body. In response to being able to sense a cardiac signal using the cardiac signal sensor, a selected pair of the electrodes from a number of electrodes positioned for stimulating a nerve is enabled for stimulation at prescribed intervals and activation levels.

### TECHNICAL FIELD

The invention relates generally to respiratory and cardiorespiratory support devices and, in particular, to an apparatus and method that reduces or eliminates a patient from exposure to a mechanical ventilator.

### BACKGROUND

Diseases, accidents, ballistic projectiles and traumas that injure high spinal cord or brain impede spontaneous respiration and cardiac rhythm lead to immediate mortality within few minutes. Although introduction of cardiorespiratory support by attending public and by trained medical personnel reduces this risk, the mortality can be still very high. Artificial ventilation using mechanical ventilators had been used to provide respiratory support in such cases and in cases where patient suffers from atelectasis, acute respiratory distress syndrome, asthma attack, chronic obstructive pulmonary disease, sepsis and the like. Even the short term use of mechanical ventilation has complications, during the first five days after the initial insult almost 80% of the deaths are caused by respiratory problems and 60% of the ICU costs are associated with it. Long term use of mechanical ventilation is not better. Mechanical ventilation not only impedes patient's quality of life (reduced mobility, sense of smell and speech) but also is the cause of respiratory complications such as atrophy of the diaphragm, reduced pulmonary function and pneumonia. It is of interest to the clinician and to the patient to reduce or eliminate exposure to mechanical ventilation as much as possible to reduce these risks.

Several noninvasive stimulation instruments that help respiration through noninvasively pacing the phrenic nerves or the heart are generally described in U.S. Pat. Nos. 3,077,884, 6,213,960, 6,312,399 and in U.S. Pat. Application Nos. 2011/0190845 and 2011/0087301, the complete disclosures are herein incorporated by reference.

Stimulation of the phrenic nerve externally could induce cardiac arrhythmias, which may be serious and potentially life-threatening. The placement of cuff electrodes around the phrenic nerves is not an option by the trained medical personnel. The provision of reliable and sufficient artificial respiration and heart beat to effectively resuscitate the patient remains a challenge. A need remains for method and associated apparatus for safely and effectively delivering phrenic nerve stimulation for respiration therapies and effectively delivering cardiac stimulation for pacing therapies.

### SUMMARY OF THE INVENTION

The aforementioned needs are addressed by the apparatus and method disclosed herein.

In one aspect of the invention, a system for providing respiratory support is disclosed.

1. The system includes an elongate body including a plurality of paired neurostimulation electrodes thereon, said electrodes configured to deliver energy to an area of tissue proximate a right phrenic nerve, a left phrenic nerve or both; monitoring means for monitoring a respiration amplitude of a patient; and a controller configured to enable the transmission of energy from the paired electrodes to the tissue proximate the right or left phrenic nerve or both, said controller adapted to
(i) select a first electrode pair of said plurality of neurostimulation electrodes;
(ii) transmit a signal to said first electrode pair to stimulate said tissue proximate said phrenic nerve; and
(iii) receive a monitoring signal from said monitoring means indicating the monitored respiration amplitude of the patient.

Other aspects of the invention are set forth in the numbered clauses that follow:

2. The system of clause 1 further comprising (iv) if said monitoring signal is indicative of an affirmative respiration amplitude, continue to transmit a signal to said first electrode pair to stimulate said tissue proximate said phrenic nerve to enable respiratory support.

3. The system of clause 1 further comprising (v) if said signal is not indicative of an affirmative respiration amplitude, transmit a signal to a third pair of electrodes; receive a monitoring signal from said monitoring means indicative of the monitored respiration amplitude of the patient; if said signal is indicative of an affirmative respiration amplitude, continue to transmit a signal to said third pair of electrodes to stimulate said tissue proximate said phrenic nerve to enable respiratory support; and if said monitoring signal is not indicative of an affirmative respiration amplitude, transmit a signal to another pair of electrodes until an affirmative respiration amplitude is received.

4. The system of clause 1 wherein said elongate body is selected from a catheter having a length of from 16 to 30 cm or from 45 to 65 cm.

5. The system of clause 4 wherein said catheter has a diameter from between 4 French to 14 French.

6. The system of clause 1 wherein said plurality of paired electrodes comprise between 2 and 32 electrodes positioned along a portion said elongate body in a spaced-apart relationship.

7. The system of clause 1 wherein said elongate body includes one or more lumens therewithin for receiving a guidewire, one or more injected drugs or saline, or for sampling blood.

8. The system of clause 1 wherein said elongate body further includes an inflatable flow directed balloon adapted to move the catheter and occlude a branch of the pulmonary artery.

9. The system of clause 1 further comprising one or more pressure sensors positioned on said elongate body and adapted to measure venous, cardiac, pulmonary artery and wedge pressures and one or more temperature sensors adapted to measure blood and injected material temperature.

10. The system of clause 1 further comprising a plurality of cardiac pacing and sensing electrodes positioned on said elongate body and adapted to deliver stimulation energy to the heart to pace the chambers of the heart and to measure electrocardiogram.

11. The system of clause 1 wherein the signal is selected from a current amplitude in the range of about 1 to about 20 milliampere; a voltage amplitude in the range of about 1 volts to about 8 volts; a frequency in the range of about 10 to about 100 Hertz (Hz); a pulse width in the range of about 20 to about 400 microseconds; a duty cycle in the range of about 300 ms to 2500 ms; and combinations of the foregoing.

12. The system of clause 1 further comprising one or more of a circuit to sense cardiac electrogram; a circuit to measure blood pressure in the hearts chambers and in the vein; a circuit to measure blood temperature; and a circuit to measure electrical impedance between a selected electrode pair of the plurality of electrodes.

13. The system of clause 1 wherein said controller is configured to (i) determine a start condition for selecting said pair of electrodes; (ii) direct electrical stimulation waveforms to said selected electrodes; and (iii) determine a stop condition to deactivate the selected electrodes.

14. The system of clause 13 wherein said start condition for selection of the electrodes is selected from time measured by a clock; a user input; detection of cardiac or respiratory activity; or a combination of the any of the foregoing.

15. The system of clause 13 wherein said direct electrical stimulation waveforms to said selected electrodes includes selection of proximal pairs of electrodes corresponding to capture of the left phrenic nerve; selection of distal pairs of electrodes corresponding to capture of right phrenic nerve; and selection of proximal and distal pairs of electrodes corresponding to capture of left phrenic nerve and right phrenic nerve.

16. The system of clause 13 wherein said determine a stop condition to deactivate the selected electrodes includes time measured by a clock; a user input; detection of cardiac or respiratory activity; or a combination of the any of the foregoing.

17. The system of clause 16 wherein the detection of respiratory activity includes a change in the electrical impedance between a selected electrode pair of said plurality of electrodes corresponding to respiratory activity; a change in the pressure corresponding to respiratory activity; or a change in the temperature corresponding to respiratory activity.

18. The system of clause 16 wherein the detection of cardiac activity includes a change in the electrical impedance between a selected electrode pair of the plurality of electrodes corresponding to cardiac activity; a change in the blood pressure corresponding to cardiac activity; or a change in the temperature corresponding to cardiac activity.

19. The system of clause 1 further comprising a cardiac signal sensing circuit, wherein said controller is configured to determine whether a cardiac signal is sensed by the cardiac signal sensing circuit by a most distal cardiac sensor positioned in a first position and if said cardiac signal is sensed enabling stimulation of the nerve using a selection of a first bipolar electrode pair in the first position.

20. The system of clause 19 wherein the controller is further configured to select a second bipolar pair of electrodes from the plurality of electrodes in response to sensing a cardiac signal.

21. The system of clause 20 wherein the second bipolar pair of electrodes is configured to stimulate a second nerve.

22. The system of clause 10 wherein the stimulation energy is selected from a pulse width between 0.05 and 5 ms, has an amplitude between 0.5 to 5 volts and has a repetition rate between 40 and 120 beats/minute; and combinations of the foregoing.

23. The system of clause 19 wherein the controller is further configured to schedule nerve stimulation pulses to be delivered using an electrode pair selected from the plurality of electrodes;
determine an electrical impedance between the first bipolar electrode pair of the plurality of electrodes in response to a stimulation of a nerve; and
switch to another electrode pair selected from the plurality of electrodes in response to changes in the electrical impedance to the stimulation of the nerve.

24. A system for providing respiratory support comprising:
a controller;
an elongate body including a plurality of paired neurostimulation electrodes lead connected to the controller;
means for stimulating phrenic nerve tissue;
means for modulating respiration in response to stimulating phrenic nerve stimulation; and
means for dosing the phrenic nerve stimulation.

25. The system of clause 24 wherein said means for dosing is configured to provide dosing on a periodic basis, upon user activation, upon user command, or in response to programmed parameters.

26. The system of clause 24 wherein the programmed parameters comprise stimulation energy.

27. The system of clause 25 wherein the programmed parameters comprise electrode selection.

28. The system of clause 25 wherein the programmed parameters comprise time measured by a clock.

### BRIEF DESCRIPTION OF THE DRAWINGS

FIG. 1A is a schematic view of a system including a respiratory support device (RD) and a respiratory support Lead (RL) for delivering respiratory support therapy according to an embodiment.

FIG. 1B is a schematic view of a system including both cardiac and respiratory support device (CRD) and a cardiac and respiratory support lead (CRL) for delivering both cardiac and respiratory (cardiorespiratory) support to a patient.

FIG. 2A is a schematic view of a system containing a RD and a RL for delivering respiratory support therapy according to an alternative embodiment.

FIG. 2B is a schematic view of a system containing a CRD and a CRL for delivering both cardiac and respiratory (cardiorespiratory) support therapy to a patient according to an alternative embodiment.

FIG. 3A is a schematic view of a RL for delivering respiratory support therapy according to one embodiment.

FIG. 3B is a schematic view of a CRL for delivering cardiorespiratory support therapy according to one embodiment.

FIG. 4 is a schematic view of a CRL for delivering cardiorespiratory support therapy according to another embodiment.

FIG. 5 is a schematic view of a CRL for delivering cardiorespiratory support therapy according to alternative embodiment.

FIG. 6A is a functional block diagram of a RD that may be associated with any of the RDs and RLs shown in FIGS. 1 through 3.

FIG. 6B is a functional block diagram of a CRD that may be associated with any of the CRDs and CRLs shown in FIGS. 1 through 5.

FIG. 7 is a flow chart of a method for positioning an RL or a CRL according to one embodiment.

FIG. 8 is a flow chart of a method for providing respiratory or cardiorespiratory support therapy according to one embodiment.

FIG. 9 is an exemplary operation of a method and apparatus for weaning from mechanical ventilator while providing respiratory support therapy according to one embodiment.

FIG. 10 is a flow chart of a method for weaning from mechanical ventilator while providing respiratory support therapy according to one embodiment.

FIG. 11 depicts a variety of parameters that may be utilized in weaning a patient from a mechanical ventilator according to one aspect of the invention.

### DETAILED DESCRIPTION

In the following description, references are made to illustrative embodiments. It is understood that other embodiments may be utilized without departing from the scope of the disclosure.

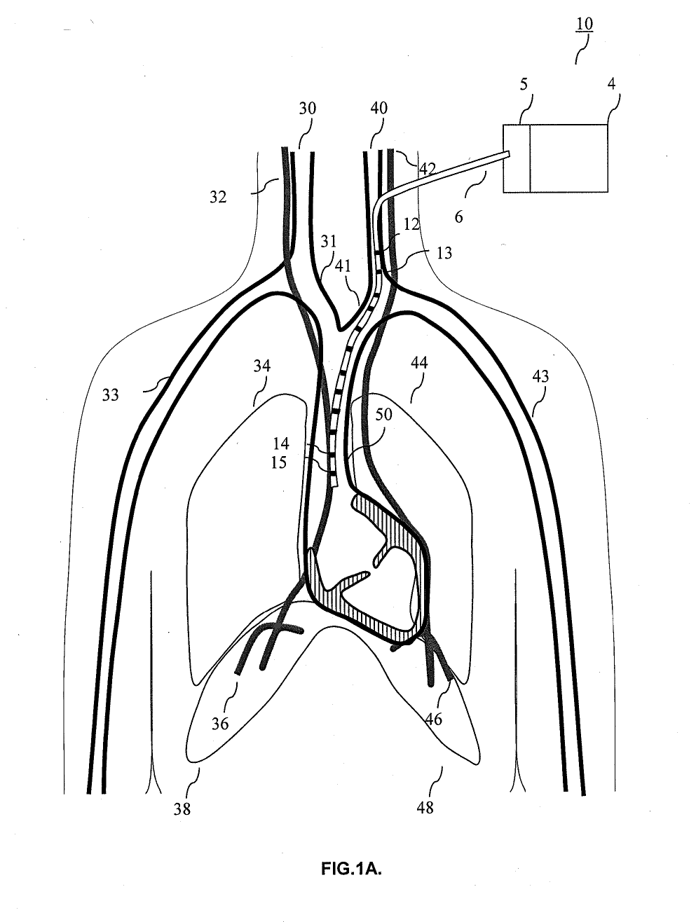

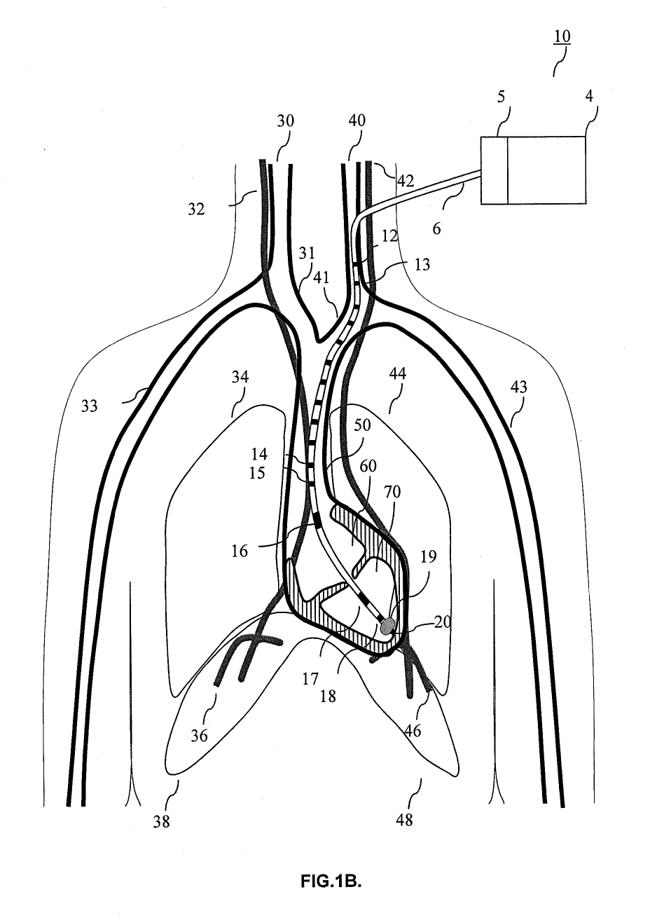

Referring generally to FIGS. 1A and 1B a system in accordance with the invention is shown. FIG. 1A is a schematic view of a system using a respiratory support device (RD) and a respiratory support lead (RL) for delivering phrenic nerve stimulation through a incision made in the left jugular vein 40. Those of skill in the art will appreciate that the system may be modified as shown in FIG. 1B to include cardiac support at the same time as supplying respiratory support. Thus, FIG. 1B is a schematic view of a system using a cardiorespiratory support device (CRD) and a cardiorespiratory support Lead (CRL) for delivering phrenic nerve stimulation through a incision made in the left jugular vein 40. For ease of convenience the inventors will refer to the CRD and CRL in this disclosure although an RD and RL are within the intended scope of the invention.

CRD 10 includes a housing 4 enclosing electronic circuitry (not shown) included in CRD 10 and a connector block 5 having a connector bore for receiving at least one CRL 6 and providing electrical connection between electrodes carried by CRL 6 and CRD 10 internal electronic circuitry.

FIGS. 1A-1B, the left phrenic nerve 42 and the right phrenic nerve 32 are shown innervating the respective left diaphragm 48 through left phrenic nerve endings 46 and right diaphragm 38 through right phrenic nerve endings 36 to cause inspiration through the left lung 44 and right lung 34. The anatomical locations of the left phrenic nerve 42, the right phrenic nerve 32 and other anatomical structures shown schematically in the drawings presented herein are intended to be illustrative of the approximate and relative locations of such structures. These structures are not necessarily shown in exact anatomical scale or location. The superior vena cava (SVC) 50, right atrium (RA) 60 and the right ventricle (RV) 70 are shown schematically in a partially cut-away view.

The anatomical location of the right phrenic nerve 32 is shown schematically to extend in close proximity to the right internal jugular vein (RJV) 30 and the right subclavian vein (RSV) 33, the right innominate vein (RIV) 31 (also referred to as the right brachiocephalic vein), and the SVC 50. The right phrenic nerve 32 extends posteriorly along the SVC 50, the RA 60 and the inferior vena cava (IVC) (not shown in FIG. 1) and descends into right diaphragm 38 through right phrenic nerve endings 36.

The left phrenic nerve 42 is shown schematically to extend in close proximity to the left internal jugular vein (LJV) 40, the left subclavian vein (LSV) 43 and the left innominate vein (LIV) 41 (also referred to as the left brachiocephalic vein). The left phrenic nerve 42 normally extends along a left lateral wall of the left ventricle (not shown) and descends into left diaphragm 48 through left phrenic nerve endings 46.

CRL 6 is a multipolar electrode array carrying proximal electrodes 12, 13 spaced proximally from distal electrodes 14, 15, positioned near the distal end 20 of CRL 6. In one embodiment, at least one proximal bipolar pair of electrodes 12, 13 is provided for stimulating the left phrenic nerve 42 and at least one distal bipolar pair of electrodes 14, 15 is provided for stimulating the right phrenic nerve 32. In various embodiments, two or more electrodes may be spaced apart along the lead body, near the distal electrode 15 of CRL 6, from which at least one pair of electrodes is selected for delivering stimulation to the right phrenic nerve 32. Additionally, two or more electrodes may be positioned along spaced apart locations proximally from the proximal electrode 12 from which at least one pair of electrodes is selected for delivering stimulation to the left phrenic nerve 42.

Distal electrode 20 of CRL 6 is shown to be advanced to a location along the RA 60 and further along the RV 70 to position distal electrode 20 to RV apex for delivering stimulation pulses to activate the RV 70. A proximal electrode 18 may be appropriately spaced from distal electrode 20 such that proximal electrode 18 is position in the RV 70 for delivering bipolar stimulation pulses to the RV 70.

In various embodiments, CRL 6 may carry a pressure sensor 16 to measure the pressure in the SVC 50 and in the RA 60 and a pressure sensor 17 to measure the pressure in the RV 70. In other embodiments, CRL 6 may carry a saline filled balloon 19 to drag the CRL 6 into the RV 70 using the flow of the blood. It should be noted that the advancement of a CRL toward the CRL may include the use of a guide catheter and/or guide wire. The CRL 6 may be an “over the wire” type lead that includes an open lumen for receiving a guide wire, over which the lead is advanced for placement at a desired location. Alternatively, the CRL may be sized to be advanced within a lumen of a guide catheter that is then retracted. Furthermore, it is recognized that in some embodiments, multiple electrodes spaced equally along a portion of the body of CRL 6 can be provided such that any pair may be selected for right phrenic nerve stimulation and any pair may be selected for left phrenic nerve stimulation based on the relative locations of the electrodes from the nerves.

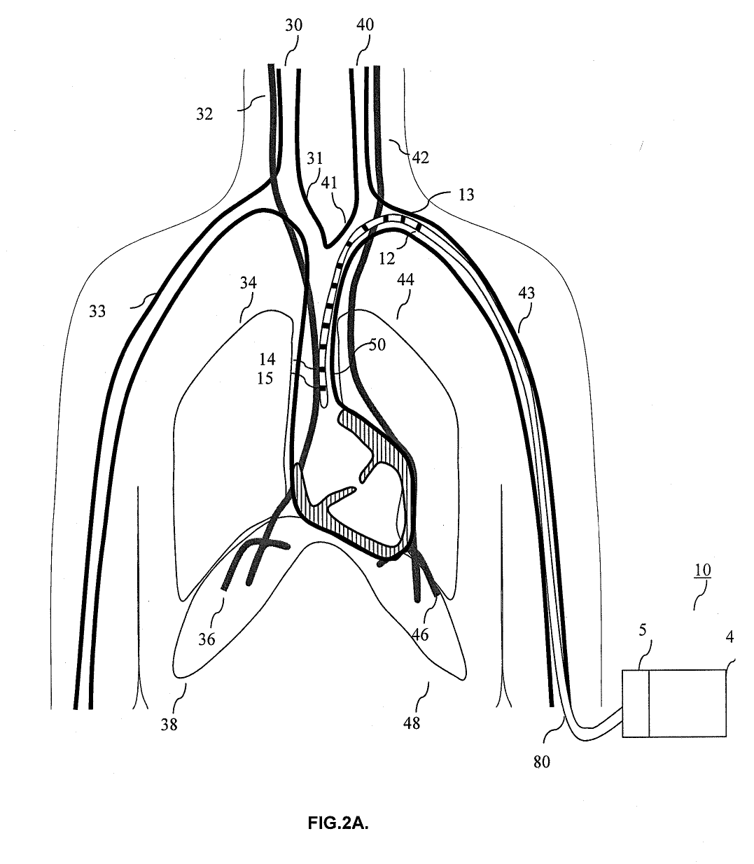

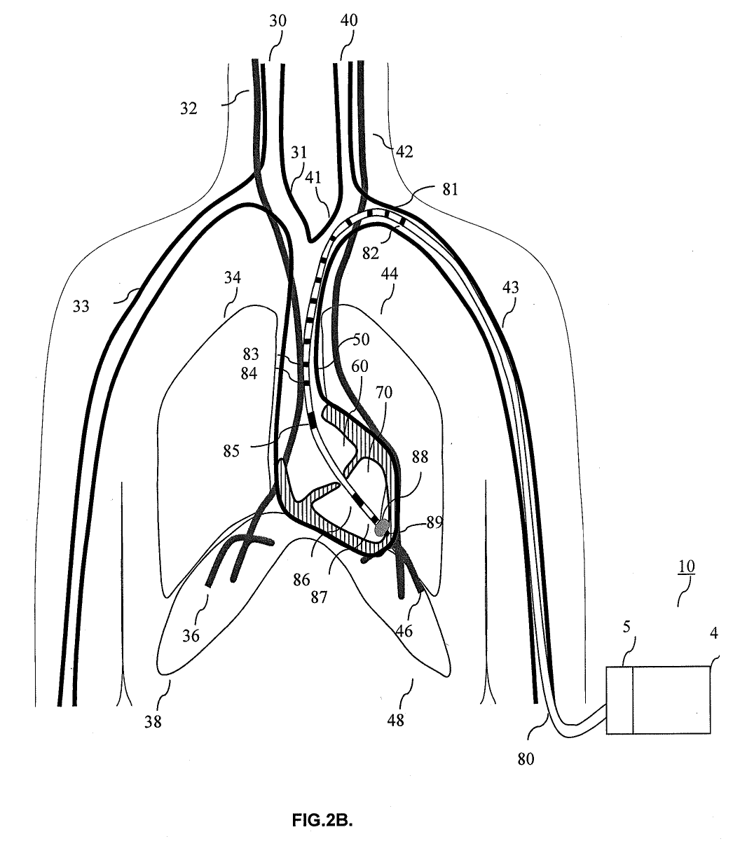

FIGS. 2A and 2B are schematic views of a system containing an RD and an RL and a CRD and a CRL, respectively, for delivering phrenic nerve stimulation according to an alternative embodiment. Those of skill in the art will appreciate that the system may be modified as shown in FIG. 2B to include cardiac support at the same time as supplying respiratory support. Thus, FIG. 2B is a schematic view of a system using a cardiorespiratory support device (CRD) and a cardiorespiratory support Lead (CRL) for delivering phrenic nerve stimulation through a incision made in the left jugular vein 40. For ease of convenience the inventors will refer to the CRD and CRL in this disclosure although an RD and RL are within the intended scope of the invention.

A CRL 80 is a multipolar electrode array carrying proximal electrodes 81, 82 spaced proximally from distal electrodes 83, 84, positioned near the distal end 89 of CRL 80. In one embodiment, at least one proximal bipolar pair of electrodes 81, 82 is provided for stimulating the left phrenic nerve 42 and at least one distal bipolar pair of electrodes 83, 84 is provided for stimulating the right phrenic nerve 32. In various embodiments, two or more electrodes may be spaced apart along the CRL 80 body, near the distal electrode 84 of CRL 80, from which at least one pair of electrodes is selected for delivering stimulation to the right phrenic nerve 32. Additionally, two or more electrodes may be positioned along spaced apart locations proximally from the proximal electrode 81 from which at least one pair of electrodes is selected for delivering stimulation to the left phrenic nerve 42.

Distal electrode 89 of CRL 80 is shown to be advanced to a location along the RA 60 and further along the right ventricle RV 70 to position distal electrode 89 to RV apex for delivering stimulation pulses to activate the RV 70. A proximal electrode 87 may be appropriately spaced from distal electrode 89 such that proximal electrode 87 is position in the RV 70 for delivering bipolar stimulation pulses to the RV 70.

In various embodiments, CRL 80 may carry a pressure sensor 85 to measure the pressure in the SVC 50 and the RA 60 and a pressure sensor 86 to measure the pressure in the RV 70. In other embodiments, CRL 80 may carry a saline filled balloon 88 to drag the CRL 80 into the RV 70 using the flow of the blood in to the RV. Furthermore, it is recognized that in some embodiments, multiple electrodes spaced equally along a portion of the body of CRL 80 can be provided such that any pair may be selected for right phrenic nerve stimulation and any pair may be selected for left phrenic nerve stimulation based on the relative locations of the electrodes from the nerves.

The RL and CRL may have a plurality of lumens that can be used to deliver drugs, sample blood, measure pressure and accommodate a guide wire. For each lumen a port hole can be provided (not shown) at appropriate distances to allow communication with the blood in the anatomical structures such as subclavian veins 43, 44, innominate veins 31, 41, vena cava 50, RA 60, RV 70, or pulmonary arteries. The CRL 80 may have a plurality of specialized connectors at the most proximal end that can be used to couple to syringes, fluid lines, pressure sensors and the like.

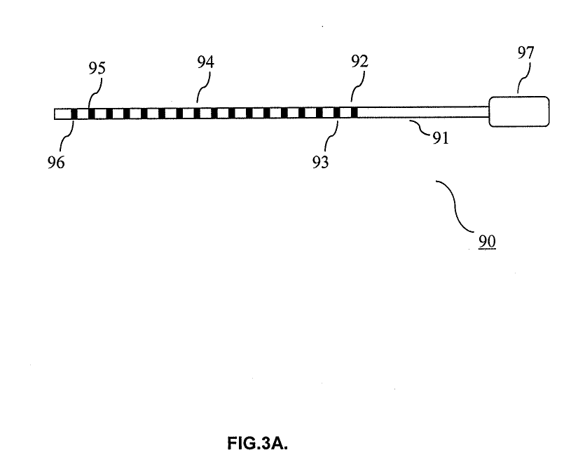

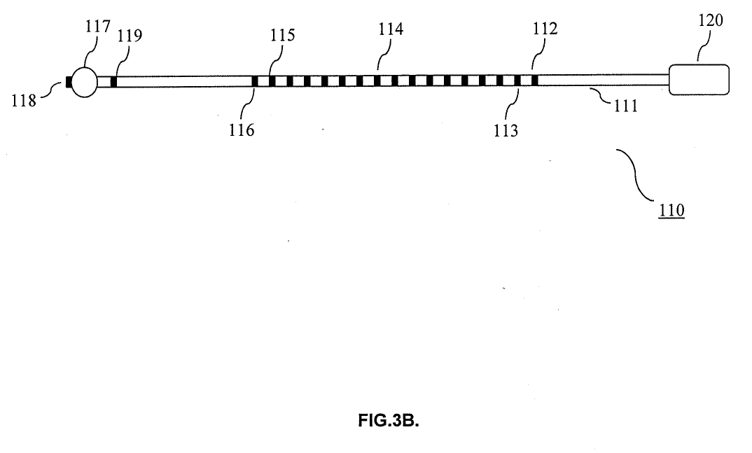

FIG. 3A is a schematic view of a RL for delivering cardiorespiratory support therapy according to one embodiment. RL 90 includes an elongated lead body 91, which may have a diameter in the range of approximately 2 French to 14 French, and typically approximately 4 French to approximately 6 French. The lead body 91 might have a length of 20 cm to 160 cm, and typically approximately 25 cm to 65 cm. The RL body 91 carries a plurality of lumens (not shown) that would be used for injecting drugs, sampling blood or measuring pressure. These lumens could terminate with openings in the RL body 91 and may have a plurality of specialized connectors next to the connector assembly 97 that can be used to couple to syringes, fluid lines and the like. In addition, RL body 91 carries proximal phrenic nerve stimulation electrodes 92, 93 and distal phrenic nerve stimulation electrodes 95, 96. It is further recognized that additional electrodes may be included in a RL 90 for delivering cardiorespiratory support therapy.

The lead body 91 might carry a plurality of phrenic nerve stimulation electrodes 94 that number in the range of 2 to 30 between the most proximal phrenic nerve stimulation electrode 92 and most distal phrenic nerve stimulation electrode 96, and typically number approximately between 6 and 14. The nerve stimulation electrodes that are carried by the lead body 91 are electrically coupled to electrically insulated conductors extending from respective individual electrodes to a proximal connector assembly 97 including connectors that enable either direct connection to RD 10 connector block 5, or via a cable with a female connector portion for receiving connector assembly 97. Alternatively, RL 90 may be configured for direct coupling to a RD 10.

Any of phrenic nerve stimulation electrodes 94 may be used for delivering a drive current and measuring a resulting impedance signal by coupling the drive and measurement electrode pairs to an impedance measuring circuit. Examples of impedance measurement methods that can be used for impedance signal are generally described in U.S. Pat. No. 4,901,725 (Nappholz), U.S. Pat. No. 6,076,015 (Hartley), and U.S. Pat. No. 5,824,029 (Weijand, et al), all of which are hereby incorporated herein by reference in their entirety.

The RL 90 can be used by positioning it in a vein of the patient through an incision made in the dermis of the patient and an introducer or other appropriate mechanism can be used to introduce the RL 90 into the vein. For example, the RL 90 can be introduced into the patient through one of the jugular veins 30, 40 as shown in FIG. 1, through one of the subclavian veins 33, 43 as shown in FIG. 2 or through any other vein in the body. It should be noted that the advancement of RL 90 toward the heart may include the use of a guide catheter and/or guide wire. The RL 90 may be an “over the wire” type that includes an open lumen for receiving a guide wire, over which the lead is advanced for placement at a desired location. Alternatively, the RL may be sized to be advanced within a lumen of a guide catheter that is then retracted.

The phrenic nerve stimulation electrodes of the RL shown in FIG. 3A can be used in pairs to measure an electrical impedance of between them. As discussed further herein, the measurement of an electrical impedance can be used to identify presence or absence of respiration, cardiac activity and to identify various regions of the venous system. In this regard, an increase or change in electrical impedance with the distal pairs 95, 96 can be used to identify regions of the venous system such as the subclavian vein, innominate vein, superior vena cava or the right atrium. The monitoring of the electrical impedance with the distal pairs can be used to identify the presence of presence of cardiac activity to control the operation of the RD 10. The monitoring of the electrical impedance with the more proximal pairs can be used to identify the presence of induced or spontaneous respiration and the presence of cardiac component to control the operation of the RD 10.

FIG. 3B is a schematic view of a CRL for delivering cardiorespiratory support therapy according to one embodiment. CRL 110 includes an elongated lead body 111, which may have a diameter in the range of approximately 2 French to 14 French, and typically approximately 4 French to approximately 8 French. The lead body 111 might have a length of 20 cm to 160 cm, and typically approximately 25 cm to 65 cm. The lead body 111 carries proximal phrenic nerve stimulation electrodes 112, 113 and distal phrenic nerve stimulation electrodes 115,116. It is further recognized that additional electrodes may be included in a CRL 110 for delivering cardiorespiratory support therapy.

The lead body 111 might carry a plurality of phrenic nerve stimulation electrodes 114 that number in the range of 2 to 30 between the most proximal phrenic nerve stimulation electrode 112 and most distal phrenic nerve stimulation electrode 116, and typically number approximately between 6 and 14. The nerve stimulation electrodes that are carried by the lead body 111 are electrically coupled to electrically insulated conductors extending from respective individual electrodes to a proximal connector assembly 120 including connectors that enable either direct connection to CRD 10 connector block 5, or via a cable with a female connector portion for receiving connector assembly 120. Alternatively, CRL 110 may be configured for direct coupling to a CRD 10. The lead body 111 carries also a proximal 119 and a most distal cardiac stimulation electrode 118 to stimulate the heart in either unipolar or bipolar configuration. The cardiac stimulation electrodes 118 and 119 are also electrically coupled to electrically insulated conductors extending from respective individual electrodes to the proximal connector assembly 120 adapted for connection to CRD connector block 5. Alternatively, a separate connector could be provided (not shown) for the cardiac stimulation electrodes 118 and 119 that may be configured for direct coupling to an external pacemaker.

Any of phrenic nerve stimulation electrodes 114 and cardiac stimulation electrodes may be used for delivering a drive current and measuring a resulting impedance signal by coupling the drive and measurement electrode pairs to an impedance measuring circuit.

The CRL shown in FIG. 3B includes various portions, such as a balloon or inflatable portion 117. The inflatable or expandable portion 117 can assist in assuring that the CRL does not puncture or perforate a wall of the RV 70 or other blood vessel. The balloon portion 117 can also act as a stop when the CRL 110 is being moved through the RV 70 or other anatomical portion. The balloon portion 117 can be inflated or deflated as selected by the user or automatically by the CRD. Inflation of the balloon portion 117 can be performed in any appropriate manner such as directing a fluid, such as a liquid or gas, through a lumen in the CRL body 111. In addition, the CRL 110 can be moved relative to the anatomy via anatomical forces placed upon various portions of the CRL 110, such as a drag created on the balloon portion 117 by the flow of blood.

The CRL 110 can be used by positioning it in a vein of the patient through an incision made in the dermis of the patient and an introducer or other appropriate mechanism can be used to introduce the CRL 110 into the vein. Once the CRL is in the vein, the balloon 117 is inflated and drag is induced on the balloon 117, due to the flow of blood in the patient. This can assist the balloon 117 to move generally in the direction of the flow of blood in the patient and allow for ease of movement and guiding of the balloon catheter 117 within the patient. For example, the CRL 110 can be introduced into the patient through one of the jugular veins 30, 40 as shown in FIG. 1B, through one of the subclavian veins 33, 43 as shown in FIG. 2B or through any other vein in the body. The flow of blood can direct the CRL 110, into the RV through the vein into SVC 50 and RA 60 towards the RV septum. In addition, the CRL 110 may be provided with a fixation element for fixing the position of the CRL once a desired implant location is identified.

A plurality of lumens can be provided within the CRL body 111 for injecting drugs, sampling blood, measuring pressures and accommodating a guidewire. These lumens could terminate with an opening in the CRL body 111 at predetermined anatomical locations. Separate connecting ports (not shown) next to the connector block 120 could be provided for interfacing lumens within the CRL body 111 to external devices such as syringes, sensors, fluid lines etc.

The phrenic nerve stimulation electrodes of the CRL shown in FIG. 3B can be used in pairs to measure an electrical impedance of between them. As discussed further herein, the measurement of electrical impedance can be used to identify presence or absence of respiration and to identify various regions of the heart. In this regard, an increase or change in electrical impedance with the distal pairs 118, 119 can be used to identify regions of the heart such as the right atrium, right ventricle, pulmonary artery, and the locations of valves. The monitoring of the electrical impedance with the more proximal pairs can be used to identify the presence of induced or spontaneous respiration and the presence of cardiac component to control the operation of the CRD 10. In addition, the cardiac stimulation electrodes 118 and 119 may additionally be used for sensing cardiac electrical signals (EGM) signals.

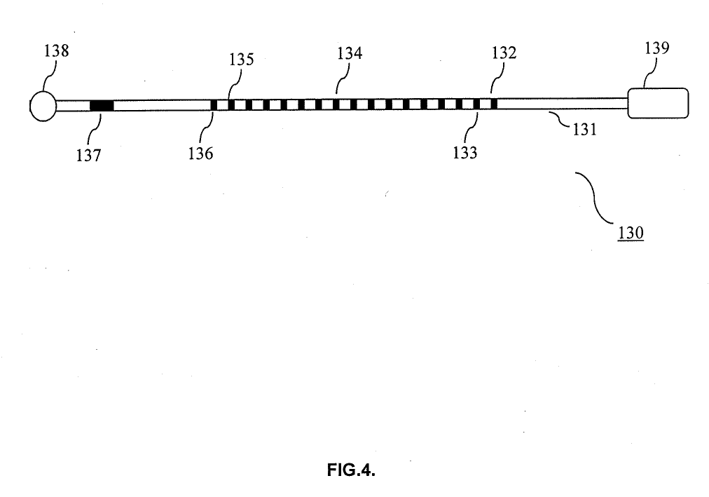

FIG. 4 is a schematic view of a CRL for delivering cardiorespiratory support therapy according to another embodiment. CRL 130 includes an elongated lead body 131, which may have a diameter in the range of approximately 2 French to 14 French, and typically approximately 4 French to approximately 8 French. The lead body 131 might have a length of 25 cm to 65 cm, and typically approximately 45 cm to 110 cm. The lead body 131 carries proximal phrenic nerve stimulation electrodes 132, 133 and distal phrenic nerve stimulation electrodes 135,136. It is further recognized that additional electrodes may be included in a CRL 130 for delivering cardiorespiratory support therapy. The lead body 131 might carry a plurality of phrenic nerve stimulation electrodes 134 that number in the range of 2 to 30 between the most proximal phrenic nerve stimulation electrode 132 and most distal phrenic nerve stimulation electrode 136, and typically number approximately between 6 and 14. The nerve stimulation electrodes that are carried by the lead body 131 are electrically coupled to electrically insulated conductors extending from respective individual electrodes to a proximal connector assembly 139 adapted for connection to CRD 10 connector block 5.

The CRL shown in FIG. 4 includes various portions, such as a balloon or inflatable portion 138. The inflatable or expandable portion 138 can assist in assuring that the CRL does not puncture or perforate a wall of the RV 70 or other blood vessel. The balloon portion 138 can also act as a stop when the CRL 130 is being moved through the RV 70 or other anatomical portion. The balloon portion 138 can be inflated or deflated as selected by the user or automatically by the CRD. Inflation of the balloon portion 138 can be performed in any appropriate manner such as directing a fluid, such as a liquid or gas, through a lumen in the CRL body 131. In addition, the CRL 130 can be moved relative to the anatomy via anatomical forces placed upon various portions of the CRL 130, such as a drag created on the balloon portion 138 by the flow of blood.

The CRL 130 can be used by positioning it in a vein of the patient through an incision made in the dermis of the patient and an introducer or other appropriate mechanism can be used to introduce the CRL 130 into the vein. Once the CRL is in the vein, the balloon 138 is inflated and drag is induced on the balloon 138, due to the flow of blood in the patient. This can assist the balloon 138 to move generally in the direction of the flow of blood in the patient and allow for ease of movement and guiding of the balloon catheter 138 within the patient. For example, the CRL 130 can be introduced into the patient through one of the jugular veins 30, 40 as shown in FIG. 1, through one of the subclavian veins 33, 43 as shown in FIG. 2 or through any other vein in the body. The flow of blood can direct the CRL 130, into the RV through the vein into SVC 50 and RA 60 towards the RV septum. In addition, the CRL 130 may be provided with a fixation element for fixing the position of the CRL once a desired implant location is identified.

A plurality of lumens can be provided within the CRL body 131 for injecting drugs, sampling blood, measuring pressures and accommodating a guidewire. These lumens could terminate with an opening in the CRL body 131 at predetermined anatomical locations. Separate connecting ports (not shown) next to the connector block 139 could be provided for interfacing lumens within the CRL body 111 to external devices such as syringes, sensors, fluid lines etc.

The phrenic nerve stimulation electrodes of the CRL shown in FIG. 4 can be used in pairs to measure an electrical impedance of between them. As discussed further herein, the measurement of electrical impedance can be used to identify presence or absence of respiration and to identify various regions of the heart. In this regard, an increase or change in electrical impedance with the distal pairs 135, 136 can be used to identify regions of the heart such as the right atrium, right ventricle, pulmonary artery, and the locations of valves. The monitoring of the electrical impedance with the more proximal pairs can be used to identify the presence of induced or spontaneous respiration and the presence of cardiac component to control the operation of the CRD 10.

The CRL shown in FIG. 4 includes a distal pressure sensor 137 to measure the pressures at a location immediately after the most distal phrenic nerve stimulation electrode 136. As discussed further herein, the measurement of a pressure pulse or a pressure change can be used to identify presence or absence of respiration and to identify various regions of the heart. In this regard, an increase or change in pulsatile pressure with the distal pressure sensor 137 can be used to identify regions of the heart such as the right atrium, right ventricle, pulmonary artery, and the locations of valves.

The pressure sensor 137 could also be more distal to the balloon 138 and can be used to measure central venous pressures, RA pressures, RV pressures, pulmonary artery or wedge pressures. These pressures could be utilized by the user to titrate various combinations of drugs and treatments. The pressure waveforms recorded in the chambers of the heart or in the pulmonary artery could be used to measure cardiac output. Alternatively the CRL could contain a thermistor (not shown) that would allow measurement of core temperature and estimation of cardiac output using thermodilution principles. The cardiac chamber pressures could also be used to estimate cardiac output.

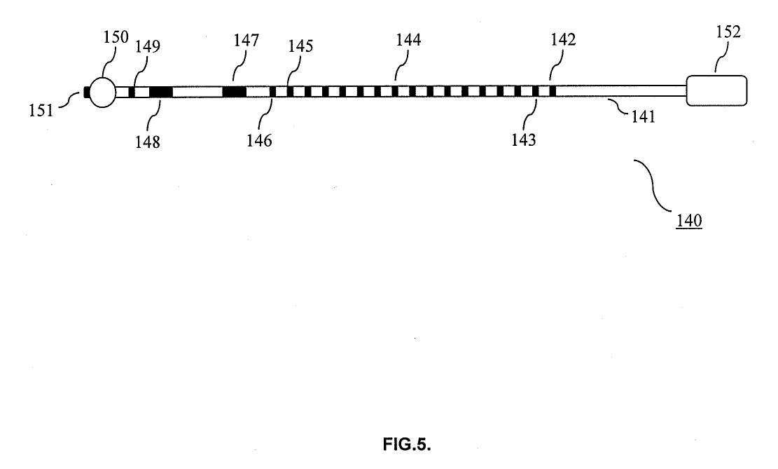

FIG. 5 is a schematic view of a CRL for delivering cardiorespiratory support therapy according to an alternative embodiment. CRL 140 includes an elongated lead body 141, which may have a diameter in the range of approximately 2 French to 14 French, and typically approximately 4 French to approximately 8 French. The CRL body 141 might have a length of 20 cm to 160 cm, and typically approximately 25 cm to 65 cm. The CRL body 141 carries proximal phrenic nerve stimulation electrodes 142, 143 and distal phrenic nerve stimulation electrodes 145, 146. It is further recognized that additional electrodes may be included in a CRL 140 for delivering cardiorespiratory support therapy. The CRL body 141 might carry a plurality of phrenic nerve stimulation electrodes 144 that number in the range of 2 to 30 between the most proximal phrenic nerve stimulation electrode 142 and most distal phrenic nerve stimulation electrode 146, and typically number approximately between 6 and 14. The nerve stimulation electrodes that are carried by the CRL body 141 are electrically coupled to electrically insulated conductors extending from respective individual electrodes to a proximal connector assembly 152 adapted for connection to CRD 10 connector block 5. The CRL body 141 carries also a proximal 149 and a most distal cardiac stimulation electrode 151 to stimulate the heart in either unipolar or bipolar configuration. The cardiac stimulation electrodes 149 and 151 are also electrically coupled to electrically insulated conductors extending from respective individual electrodes to the proximal connector assembly 152 adapted for connection to CRD 10 connector block 5. Alternatively, a separate connector could be provided (not shown) for the cardiac stimulation electrodes 149 and 151 that may be configured for direct coupling to an external pacemaker.

The CRL shown in FIG. 5 includes various portions, such as a balloon or inflatable portion 150. The inflatable or expandable portion 150 can assist in assuring that the CRL does not puncture or perforate a wall of the RV 70 or other blood vessel. The balloon portion 150 can also act as a stop when the CRL 140 is being moved through the RV 70 or other anatomical portion. The balloon portion 150 can be inflated or deflated as selected by the user or automatically by the CRD. Inflation of the balloon portion 150 can be performed in any appropriate manner such as directing a fluid, such as a liquid or gas, through a lumen in the CRL body 141. In addition, the CRL 140 can be moved relative to the anatomy via anatomical forces placed upon various portions of the CRL 140, such as a drag created on the balloon portion 150 by the flow of blood.

A plurality of lumens can be provided within the CRL body 141 for injecting drugs, sampling blood, measuring pressures and accommodating a guidewire. These lumens could terminate with an opening in the CRL body 141 at predetermined anatomical locations. Separate connecting ports (not shown) next to the connector block 152 could be provided for interfacing lumens within the CRL body 141 to external devices such as syringes, sensors, fluid lines etc.

The CRL 140 can be used by positioning it in a vein of the patient through an incision made in the dermis of the patient and an introducer or other appropriate mechanism can be used to introduce the CRL 140 into the vein. Once the CRL is in the vein, the balloon 150 is inflated and drag is induced on the balloon 150, due to the flow of blood in the patient. This can assist the balloon 150 to move generally in the direction of the flow of blood in the patient and allow for ease of movement and guiding of the CRL 140 within the patient. For example, the CRL 150 can be introduced into the patient through one of the jugular veins 30, 40 as shown in FIG. 1, through one of the subclavian veins 33, 43 as shown in FIG. 2 or through any other vein in the body. The flow of blood can direct the CRL 140, into the RV through the vein into SVC 50 and RA 60 towards the RV septum. In addition, the CRL 150 may be provided with a fixation element for fixing the position of the CRL once a desired implant location is identified.

The CRL shown in FIG. 5 includes a proximal pressure sensor 147 and a distal pressure sensor 148 to measure the pressures at a location immediately after the most distal phrenic nerve stimulation electrode 146 and immediately before the most proximal cardiac stimulation electrode 149. As discussed further herein, the measurement of a pressure pulse or a pressure change can be used to identify presence or absence of respiration and to identify various regions of the heart. In this regard, an increase or change in pulsatile pressure with the distal pressure sensor 148 can be used to identify regions of the heart such as the right atrium, right ventricle, pulmonary artery, and the locations of valves. The monitoring of the pulsatile pressures with the proximal pressure sensor 147 can be used to identify the presence of induced or spontaneous respiration and the presence of cardiac component to control the operation of the CRD 10. The pressure sensors 147 and 148 could also be used to measure central venous pressures, trans-tricuspid pressure gradient, RA pressures, RV pressures, pulmonary artery or wedge pressures. These pressures could be utilized by the user to titrate various combinations of drugs and treatments. The pressure waveforms recorded in the chambers of the heart or in the pulmonary artery could be used to measure cardiac output. Alternatively the CRL could contain a thermistor (not shown) that would allow measurement of core temperature and estimation of cardiac output using thermodilution principles. The cardiac chamber pressures could also be used to estimate cardiac output.

The phrenic nerve stimulation electrodes of the CRL shown in FIG. 5 can be used in pairs to measure an electrical impedance of between them. As discussed further herein, the measurement of an electrical impedance can be used to identify presence or absence of respiration and to identify various regions of the heart. In this regard, an increase or change in electrical impedance with the distal pairs 145, 146 can be used to identify regions of the heart such as the right atrium, right ventricle, pulmonary artery, and the locations of valves. The monitoring of the electrical impedance with the more proximal pairs can be used to identify the presence of induced or spontaneous respiration and the presence of cardiac component to control the operation of the CRD 10.

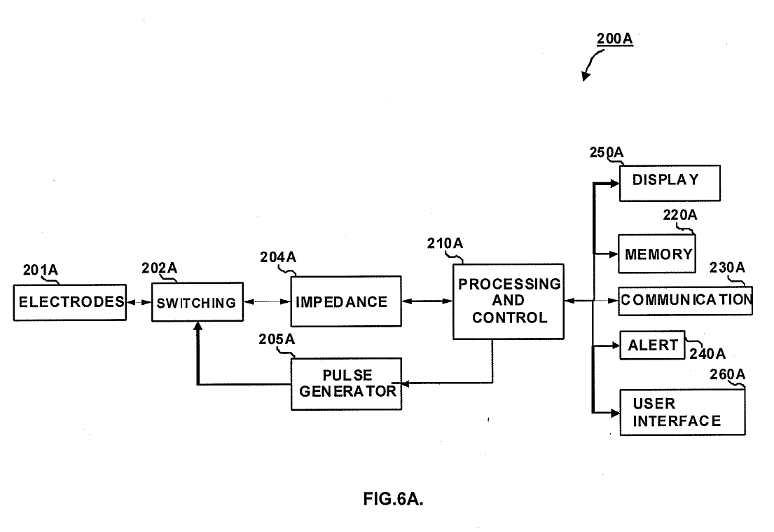

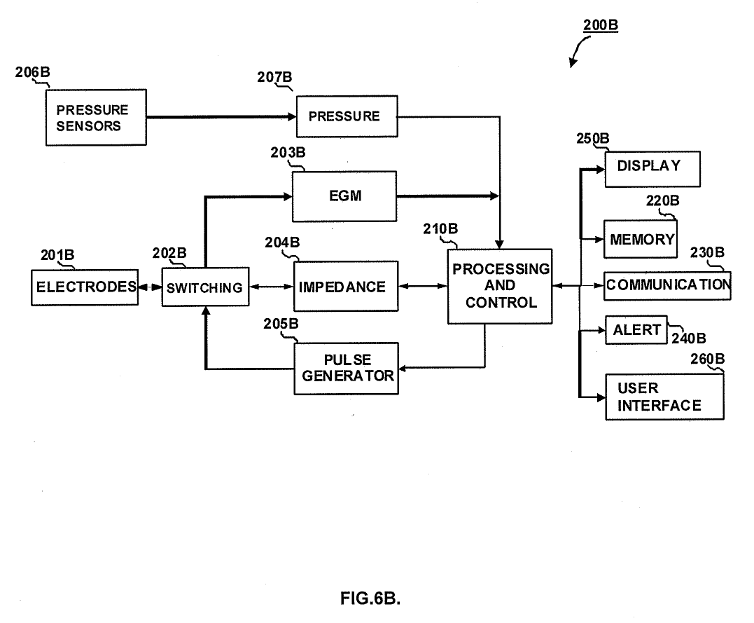

FIG. 6A is a functional block diagram 200A of a RD 10 that may include any of the RLs and implant locations shown in FIGS. 1 through 3. Electrodes 201A are coupled to impedance sensing 204A, and pulse generator 205A via switching circuitry 202A. Electrodes 201A may correspond to any of the electrodes shown in FIGS. 1 through 3.

Electrodes 201A are selected in impedance signal drive current and measurement pairs via switching circuitry 202A for monitoring electrical impedance by impedance monitoring circuitry 204A. Electrodes 201A are further selected via switching circuitry 202A for delivering phrenic nerve stimulation pulses generated by pulse generator 205A.

EGM sensing circuitry 203A is provided for sensing for the presence of an EGM signal on electrodes during nerve stimulation therapy delivery for detecting cardiac activation.

The impedance sensing circuitry 204A includes drive current circuitry and impedance measurement circuitry for monitoring electrical impedance. The electrical impedance measurements can be used to select optimal electrodes and stimulation parameters for achieving a desired effect on respiration caused by phrenic nerve stimulation. In addition, the electrical impedance is used to sense cardiac activity and to sense a respiratory response to phrenic nerve stimulation. If the electrodes are located in close proximity of the heart, phrenic nerve stimulation pulses will be delivered to the heart, potentially capturing myocardial tissue. If cardiac activity can be sensed using the electrodes, the phrenic nerve stimulation may be postponed to eliminate the risk of unintentional cardiac stimulation. In response to received signals processing and control 210A controls delivery of phrenic nerve by pulse generator 205A. Processing and control 210A may be embodied as a programmable microprocessor and associated memory 220A. Received signals may additionally include user command signals received by communication circuitry 230A from an external programming device and used to program processing and control 210A. Processing and control 210A may be implemented as any combination of an application specific integrated circuit (ASIC), an electronic circuit, a processor (shared, dedicated, or group) and memory that execute one or more software or firmware programs, a combinational logic circuit, or other suitable components that provide the described functionality.

Memory 220A stores data associated with the impedance signals. Data may be transmitted to an external device by communication circuit 230A, which typically includes wired or wireless transmitting and receiving circuitry and an associated cables or antenna for bidirectional communication with an external device. Processing and control 210A may generate reports or alerts that are transmitted by communication circuitry 230A.

Alert circuitry 240A may be provided for generating a patient alert signal to notify the user or the medical personnel of a condition warranting medical attention. In one embodiment, an alert is generated in response to sensing a cardiac activity signal or a respiration signal using phrenic nerve stimulation electrodes and/or detecting inadvertent capture of the heart. It could also provide an alert if possible RL dislodgement or arrhythmias is detected. The user or the medical personnel may be alerted via an audible sound, perceptible vibration, optical signals, a screen display or the like and be advised to seek further medical attention.

A display 250A may be provided for displaying the electrical impedance signals. In addition the display could also display the respiration signal, the therapy waveforms, the weaning regimes, alerts and other information that would be useful for user to interact using the user interface 260A. The user interface 250A consists of a mouse, a trackball, a keyboard, a touch screen, a plurality of buttons etc and would enable user to enter data, select therapy parameters, enabling and disabling therapies and the like.

FIG. 6B is a functional block diagram 200B of a CRD 10 that may include any of the CRLs and implant locations shown in FIGS. 1 through 5. Electrodes 201B are coupled to EGM sensing 203B, impedance sensing 204B, and pulse generator 205B via switching circuitry 202B. Electrodes 201B may correspond to any of the electrodes shown in FIGS. 1 through 5.

Electrodes 201B are selected via switching circuitry 202B for coupling to EGM sensing circuitry 203B to sense for the presence of EGM signals on cardiac stimulation electrodes for evidence cardiac activity. Electrodes 201B may also be selected in impedance signal drive current and measurement pairs via switching circuitry 202B for monitoring electrical impedance by impedance monitoring circuitry 204B. Electrodes 201B are further selected via switching circuitry 202B for delivering phrenic nerve stimulation pulses and/or cardiac stimulation pulses generated by pulse generator 205B.

EGM sensing circuitry 203B is provided for sensing for the presence of an EGM signal on cardiac stimulation electrodes during nerve stimulation therapy delivery for detecting cardiac activation. If the electrodes selected for phrenic nerve stimulation are located in close proximity of the heart, phrenic nerve stimulation pulses will be delivered to the heart, potentially capturing myocardial tissue. If an EGM signal can be sensed using the cardiac stimulation electrodes, and the heart rate deemed to be acceptable the cardiac stimulation may be postponed to eliminate the risk of unintentional cardiac stimulation.

The impedance sensing circuitry 204B includes drive current circuitry and impedance measurement circuitry for monitoring electrical impedance. The electrical impedance measurements can be used to select optimal electrodes and stimulation parameters for achieving a desired effect on respiration caused by phrenic nerve stimulation. In addition, the pressure sensors 206B is used to sense cardiac and to sense a respiratory response to phrenic nerve stimulation through the pressure 207B interface to the processing and control 210B unit. The processing and control unit also receives signals from EGM sensing 203B and impedance sensing circuitry 204B. In response to received signals processing and control 210B controls delivery of phrenic nerve and cardiac stimulation by pulse generator 205B. Processing and control 210B may be embodied as a programmable microprocessor and associated memory 220B. Received signals may additionally include user command signals received by communication circuitry 230B from an external programming device and used to program processing and control 210B. Processing and control 210B may be implemented as any combination of an application specific integrated circuit (ASIC), an electronic circuit, a processor (shared, dedicated, or group) and memory that execute one or more software or firmware programs, a combinational logic circuit, or other suitable components that provide the described functionality.

Memory 220B stores data associated with the monitored EGM (or ECG), pressure and impedance signals. Data may be transmitted to an external device by communication circuit 230B, which typically includes wired or wireless transmitting and receiving circuitry and an associated cables or antenna for bidirectional communication with an external device. Processing and control 210B may generate reports or alerts that are transmitted by communication circuitry 230B.

Alert circuitry 240B may be provided for generating a patient alert signal to notify the user or the medical personnel of a condition warranting medical attention. In one embodiment, an alert is generated in response to sensing an EGM signal or a respiration signal using cardiac or phrenic nerve stimulation electrodes and/or detecting inadvertent capture of the heart. It could also provide an alert if possible CRL dislodgement, arrhythmias or life threatening cardiac pressures is detected. The user or the medical personnel may be alerted via an audible sound, perceptible vibration, optical signals, a screen display or the like and be advised to seek further medical attention.

A display 250B may be provided for displaying the electrical impedance, EGM and pressure signals. In addition the display could also display the respiration signal, the therapy waveforms, the weaning regimes, alerts and other information that would be useful for user to interact using the user interface 260B. The user interface 250B consists of a mouse, a trackball, a keyboard, a touch screen, a plurality of buttons etc and would enable user to enter data, select therapy parameters, enabling and disabling therapies and the like.

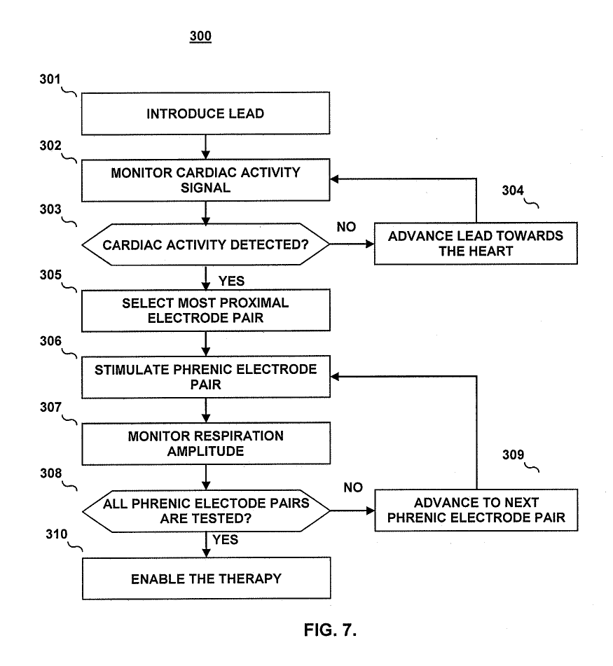

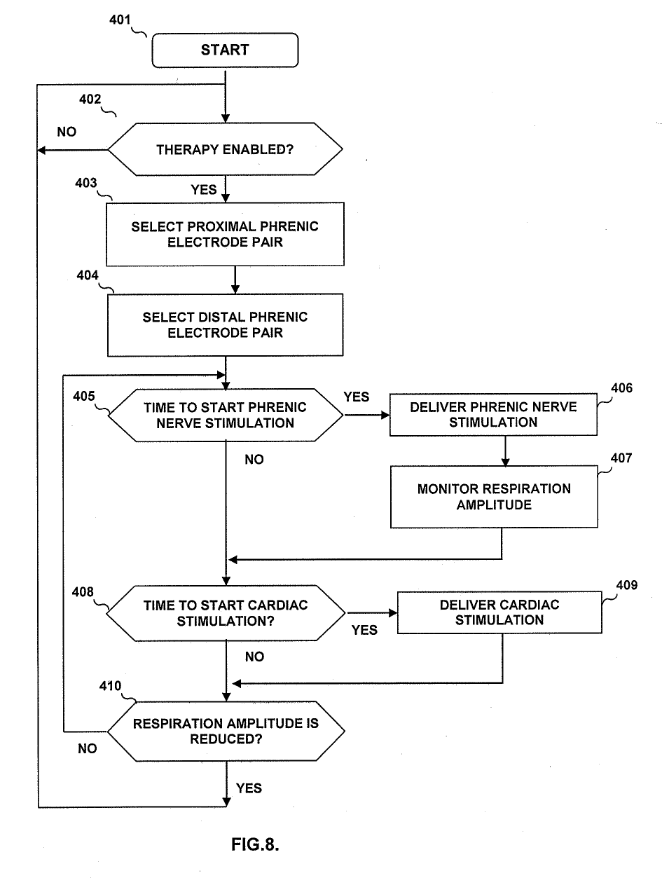

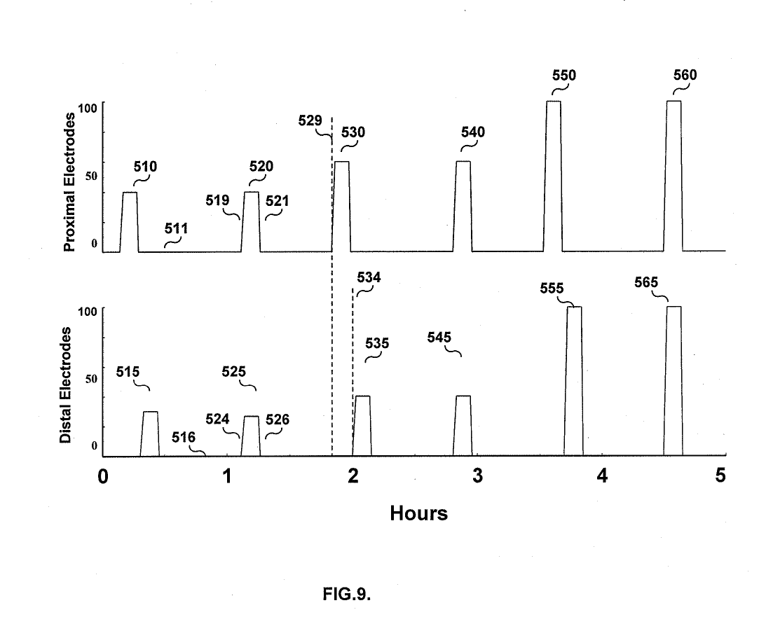

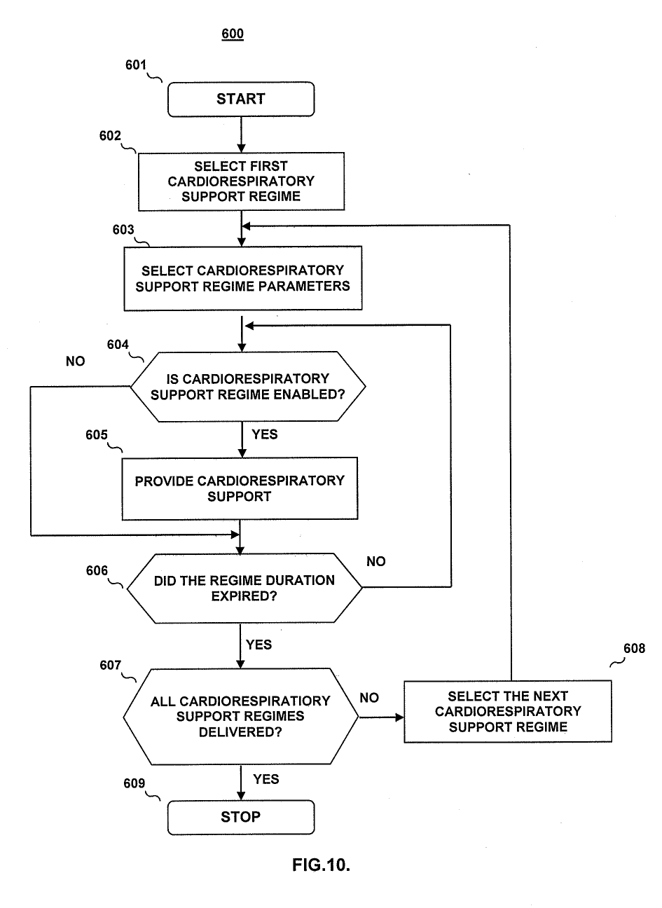

Referring generally to FIGS. 7-8 the flowcharts may apply to a system of providing respiratory support alone or providing cardiorespiratory support to a patient. Similarly, the system in FIGS. 9-10 may apply solely to the delivery of respiratory support alone or may be directed to the delivery of cardiorespiratory support.

FIG. 7 is flow chart 300 of depicting a method for positioning an RL or CRL according to one embodiment. It is recognized that the procedures described in conjunction with flow chart 300 may be performed in a different order than described here or some procedures may be omitted in a method for positioning an RL or CRL. For example, the method may include sensing for EGM signals present on phrenic electrodes using any available electrodes, or both.

An RL or CRL is introduced via a venous puncture and vein introducer device at block 301. A cardiac activity signal is monitored at block 302 and a determination is made at block 303 if the cardiac activity is detected. If the introduced lead is an RL the monitored cardiac activity signal at block 302 may be an electrical impedance signal that could be detected between cardiac electrodes 95 and 96 of FIG. 3A. A typical cardiac electrical impedance signal would be oscillatory and would have a period between 300 to 2000 milliseconds. The cardiac electrical impedance signal would have a mean value of 200 to 1500 ohms, typically 500 ohms. The pulsatile part of the cardiac electrical impedance signal would have an amplitude between 2 to 10 ohms, and more typically between 1 and 2 ohms.

If the introduced lead is a CRL the monitored cardiac activity signal at block 302 may be an electrical impedance signal that could be detected between cardiac electrodes 118 and 119 of FIG. 3B or 149 and 151 of FIG. 5. A typical cardiac electrical impedance signal would be oscillatory and would have a period between 300 to 2000 milliseconds. The cardiac electrical impedance signal would have a mean value of 200 to1500 ohms, typically 500 ohms. The pulsatile part of the cardiac electrical impedance signal would have an amplitude between 2 to 10 ohms, and more typically between 1 and 2 ohms.

The monitored cardiac activity signal at block 302 using a CRL may be an electrogram (EGM) signal that could be detected between cardiac electrodes 118 and 119 of FIG. 3B or 149 and 151 of FIG. 5. The EGM signal may be based on sensing P-waves or R-waves using a sense amplifier and auto-adjusting threshold, for example as generally described in U.S. Pat. No. 5,117,824 (Keimel, et al.), hereby incorporated herein by reference in its entirety. The rate of sensed events may be compared to an expected range of possible heart rates to indicate regular R-wave or P-wave sensing. Additionally or alternatively, a morphology analysis may be performed to compare the morphology of an unknown sensed signal to a known EGM signal morphology template to determine if the unknown morphology approximately matches the EGM signal morphology. The displayed signal may be inspected by a user instead of or in addition to an automatic signal analysis for detecting the presence of an EGM signal sensed by the phrenic nerve stimulation electrodes. In some embodiments, the EGM signal measurement at block may include a signal amplitude criterion. For example, R-wave sensing at or above a predefined sensing threshold or R-wave peak amplitudes exceeding a predefined amplitude may be required before CRL repositioning is necessary. Low level signals may indicate that the electrodes are far enough from the heart. A typical EGM signal would be oscillatory and would have a period of 300 ms to 2000 ms and amplitude between 0.3 and 30 millivolts, more typically about 1.5 millivolts.

The monitored cardiac activity signal at block 302 using a CRL may be an evoked response signal that could be detected between cardiac electrodes 118 and 119 of FIG. 3B or 149 and 151 of FIG. 5. For this purpose, a cardiac stimulation current could be passed between cardiac electrodes 118 and 119 of FIG. 3B or 149 and 151 of FIG. 5 and the resultant cardiac depolarization could be measured. Typical cardiac stimulation pulses used for this purpose would have a pulse width between 0.05 and 5 ms, have an amplitude between 0.5 to 5 volts and would have a repetition rate between 40 and 120 beats/minute.

The monitored cardiac activity signal at block 302 using a CRL may a pressure waveform measured using sensor 137 of FIG. 4 or 148 of FIG. 5. A typical cardiac pressure waveform would have a pulsatile amplitude of 6 to 100 mmHg, and more typically between 10 and 20 mmHg. The cardiac pressure would also have a period of 300 ms to 2000 ms.

At block 303 a determination was made to see if the monitored cardiac activity is indicative of cardiac contraction. If the determination was made that the monitored cardiac activity is not indicative of cardiac contraction, the RL or CRL is further advanced toward the heart at block 304, facilitated by the inflatable balloon of the CRL or facilitated by the users actions and the method returns to block 302 to keep monitoring the cardiac activity. Otherwise the method continues with block 305 in which the most proximal phrenic nerve stimulation electrodes would be selected using the switching circuits 202A of FIG. 6A for RL or 202B of FIG. 6B for CRL. The pulse generator 205A of FIG. 6A for RL or 205B of FIG. 6B for CRL would then issue a phrenic stimulation test pulse. Typical test pulse would be between 1 and 5 volts, preferably between 1 and 3 volts and more preferably 2.5 volts at block 306. Also at block 306 the phrenic stimulation test pulse would have duration between 50 and 1500 microseconds, preferably between 200 microseconds to 800 microseconds and more preferably 400 microseconds.

At block 307 a respiration amplitude is monitored during the delivery of phrenic nerve stimulation test pulse. In certain embodiments of the respiration amplitude monitoring step the electrical impedance measuring circuitry 204A or 204B of RD or CRD 10 could be engaged to measure the electrical impedance between a selected pair of phrenic nerve stimulation electrodes of the RL or CRL. The phrenic electrode pair impedance signal will be a cyclic signal that increases to a maximum during expiration as the veins are smaller and decreases to a minimum during inhalation as the veins are distended with blood producing a lower electrical impedance. A monitored respiration amplitude may be an average impedance, a maximum impedance, a maximum to minimum difference (peak-to-peak difference), a slope, an area, or other measurement correlated to respired volume, any of which may be averaged over one or more respiration cycles and taken alone or in any combination. The monitored respiration amplitude could be a change in the pre-stimulation impedance measurement and the impedance measurement obtained during the stimulation of the phrenic electrode pair. The monitored respiration amplitude may be derived as a difference or a ratio of the pre-stimulation impedance measurement and the measurement obtained during stimulation. In other embodiments of the respiration amplitude monitoring step the pressure measuring circuitry 207B of CRD 10 could be engaged to measure the pressure. A typical pressure signal correlated with the respiration will be a cyclic signal that increases to a maximum during expiration as the veins are smaller but pressurized and should decrease to a minimum during inhalation as the veins are distended with blood and the pressures are lower.

A determination is then made at block 308 if all the pairs of phrenic nerve stimulation electrodes have been utilized. If the result is not affirmative the process proceeds to block 308 where next pair of phrenic nerve stimulation electrodes are engaged using the switching circuit 202A of FIG. 6A for RL or 202B of FIG. 6B for CRL. The process then continues to block 306. If on the other hand, all phrenic nerve stimulation electrodes were utilized by the switching circuit, the method then proceeds to block 310 where RL is fixed in place and the respiratory support therapy is enabled. Alternatively, the inflatable balloon of the CRL may be deflated, the CRL is fixed in place and the cardiorespiratory support therapy is enabled. RL and CRL fixations may involve suturing or anchoring a proximal portions of the RL or CRL or the use of lead fixation members.

FIG. 8 is a flow chart 400 of a method for delivering one of a respiratory or cardiorespiratory support therapy according to one embodiment. At block 401, an RL or CRL is positioned using any of the methods described above and coupled to RD or CRD 10.

At block 402, a determination is made whether the respiratory or cardiorespiratory support therapy is enabled. In some embodiments, support therapies are started immediately upon enabling the therapy. In other embodiments, therapies may be halted or suspended temporarily and might require a user command or a user activation. If the therapies are enabled stimulation parameters for respiratory and cardiorespiratory therapies and a pair of proximal phrenic electrodes that are to be used for delivering phrenic nerve stimulation pulses are selected at block 403. Otherwise, the process continues to wait until it is time to start respiratory or cardiorespiratory support therapy as determined at block 402.

Selection of proximal phrenic electrode pairs at block 403 may involve determining the respiration amplitude in response to stimulation of the phrenic electrode pairs. The amplitude determination at block 403 may include delivering single pulses, maximum pulse energy pulses, or other stimulation pulses to selected electrodes and monitoring phrenic electrode pair impedance amplitude as generally described above. Multiple electrode pairs may be tested for phrenic electrode pair impedance amplitudes in an automated, sequential or simultaneous manner using a multi-channel impedance sensing circuit. The monitored phrenic electrode pair impedance amplitudes are analyzed for the most proximal pairs that would provide the highest phrenic electrode pair impedance amplitude.

At block 404 the distal phrenic electrode pairs that are to be used for delivering phrenic nerve stimulation pulses are selected. Again the selection of distal phrenic electrode pairs at block 404 may involve determining the phrenic electrode pair impedance amplitude or a distal pressure amplitude in response to stimulation of the phrenic electrode pairs as generally described above. The monitored phrenic electrode pair impedance amplitudes or distal pressure amplitude are analyzed using methods generally described above for the most distal pairs that would provide the highest phrenic electrode pair impedance amplitude. Alternatively, proximal and distal electrodes could be selected and presented to the block 404 as part of the cardiorespiratory regime field.

At block 405, a determination is made whether it is time to start phrenic nerve stimulation which may be scheduled to occur on a periodic basis. If it is time to start phrenic nerve stimulation, the process continues to block 406 where phrenic nerve stimulation is delivered. Otherwise the process continues with block 408. At block 406 the proximal or distal phrenic electrode pairs that were selected at blocks 403 and 404 are enabled and the phrenic nerve stimulation therapy is delivered. The typical phrenic nerve stimulation therapy consists of a therapy waveform composed of a plurality of pulses in which each pulse a pulse between 50 and 2500 microseconds ms, has amplitude between −5 to 5 volts and has a repetition rate between 10 and 100 pulses per second. The therapy waveform containing the plurality of pulses could last 0.5 to 3 seconds. The therapy waveform could be cycled every 2 to 10 seconds. Each pulse contained in the therapy waveform could be different and could be bipolar, shaped to resemble a rectangle, trapezoid, triangle, exponential rise and the like. The therapy waveform envelope could be rectangular, trapezoidal, triangular, exponential and the like. The phrenic stimulation therapy waveform envelope could be modulated by changing the frequency, amplitude, duration, pulse width and the pulse shape of the individual pulses. The resultant respiration amplitude is monitored using methods generally described above at block 407 and the process continues with block 408.

At block 408, a determination is made whether cardiorespiratory therapy is enabled and if so whether it is time to start cardiac stimulation which may be scheduled to occur on a periodic basis. If it is time to start cardiac stimulation, the process continues to block 409 where cardiac stimulation is delivered. Otherwise the process continues with block 410. At block 409 the cardiac stimulation electrodes are enabled and a cardiac stimulation pulse is delivered if there is no intrinsic cardiac electrical activation. The cardiac stimulation pulse typically has a pulse width between 0.05 and 5 ms, has an amplitude between 0.5 to 5 volts and has a repetition rate between 40 and 120 beats/minute. Once the cardiac stimulation is delivered the process continues with block 410.

At block 410 a determination is made whether the respiration amplitude is changed following the delivery of phrenic nerve stimulation. Various factors will determine whether respiration amplitude is reduced following the phrenic nerve stimulation. Such factors include the patient's dependence on phrenic nerve stimulation for respiration, blood loss or infusion, diaphragmatic fatigue, anodal stimulation, a change in the relative distance between the phrenic nerves and the phrenic nerve stimulation electrodes. For this purpose a series of monitored phrenic electrode pair impedance amplitudes or distal pressure amplitudes are compared at block 410 to determine if the last recorded value is different than a desired threshold level. A desired threshold level may be a percentage of the last recorded value and may be tailored to individual patients and will depend on the particular needs and therapy objectives for a given patient.

If a determination is made that the respiration amplitude was changed the process continues with block 402 to suspend, terminate, choose a new proximal and distal phrenic electrode pairs or select new stimulation parameters for cardiorespiratory therapy. Alternatively the process follows with block 405 to continue evaluating if it is time to start the phrenic nerve stimulation.

FIG. 9 shows an exemplary operation of a method and apparatus for weaning from mechanical ventilator using an RL or CRL according to one embodiment. In this exemplary operation it is considered that the mechanical ventilator is operating on assist mode, ie the mechanical ventilator can detect an inspiratory effort by the patient and can titrate the pressure or volume administered accordingly. The behavior of the mechanical ventilator in this and subsequent descriptions are not described but considered to be known in the art. Accordingly, a five hour weaning process using the proximal electrode pairs and distal electrode pairs is depicted. The proximal electrode pairs are activated at different 510, 530, 550 or same levels 520, 540, 560 at different times during the process of weaning to condition the section of the diaphragm innervated by the proximal phrenic nerve. The electrode activation levels in shown in FIG. 9 are scaled between 0 to 100 and indicative of the maximum deliverable therapy. The electrode activation levels could be an individual or combinatory function of the stimulus amplitude, stimulus frequency, and pulse duration or pulse shape. Between the delivery of each activation stimulation of the proximal electrodes there is given a variable time period 511 during which the proximal electrodes are not activated and the section of the diaphragm innervated by the proximal phrenic nerve is allowed to rest. This inactivated period could be between a few seconds to several hours, preferable measured in minutes. Thus the proximal nerve is activated for a brief period and given the opportunity rest between activations allowing the muscle to recover and remodel between the weaning therapies. The proximal electrodes may not be activated or deactivated instantly and can involve a train-in period 519 lasting few seconds to hours, preferably measured in minutes, during which the activation level is gradually increased. Once activation level reaches the prescribed steady level 520, 530 and etc the activation level of the proximal electrode is kept constant for a prescribed period of time preferably measured in minutes. Subsequently the activation level can be trained-out by reducing its level gradually over few seconds to few hours preferable within few minutes to zero 521. This gradual reduction allows conditioning of the muscle and elimination of waste products such as free radicals, metabolites while maintaining a steady perfusion of blood into the muscle.

During the weaning a patient from mechanical ventilator process shown in FIG. 9 the distal electrode pairs could also be activated at different 515, 535, 555 or same levels 525, 545, 565 at different times. Similar to proximal electrode pair activation pattern, the distal electrode pairs could have a steady 525, train-in 524 and/or train out 526 periods inter-dispersed with inactivated periods 516. In addition the proximal and distal electrode pairs could be activated simultaneously as shown in 520 and 525, 540 and 545, and 560 and 565. Alternatively the proximal and distal electrode pairs could be activated one 529 after the other 534. These in-phase and out of phase activation patterns help train and wean the weak portion of the diaphragm without compromising the ventilation.

FIG. 10. is a flow chart 600 of a method for weaning patients from mechanical ventilators according to one embodiment. At block 601, an RL or CRL is positioned using any of the methods described above and coupled to RD or CRD 10. The RD or CRD then executes a series of respiratory support regimes that will orderly enable a series of proximal and distal electrodes at pre-specified activation levels and durations to generate activation sequences generally described in the exemplary embodiment shown in FIG. 9.

At block 602, a first respiratory support regime is selected from a list of regimes located in memory, computer disk, internet or other medium that contains the respiratory support regime repository. At block 603 the parameters of the selected respiratory support regime is inspected. A decision is then made to see if the selected respiratory support regime is enabled at block 604. If the respiratory support regime is enabled then the process continues with block 605 otherwise the process continues with block 606. At block 605 the respiratory support regime parameters are provided to the respiratory support therapy method, the flowchart of which is given in FIG. 8. The respiratory or cardiorespiratory support is delivered using the method generally described in relation to FIG. 8. At block 606 a decision is made to assess if the respiratory support regime duration has expired and if it has not, the process continues with block 604. Otherwise the process continues with block 607 where a decision is made to assess if all the respiratory support regimes have been operated on. If the result of this decision is affirmative the process continues with block 609 where the process stops. Otherwise the next respiratory support regime from the list is selected at block 608 and the process continues with block 603.

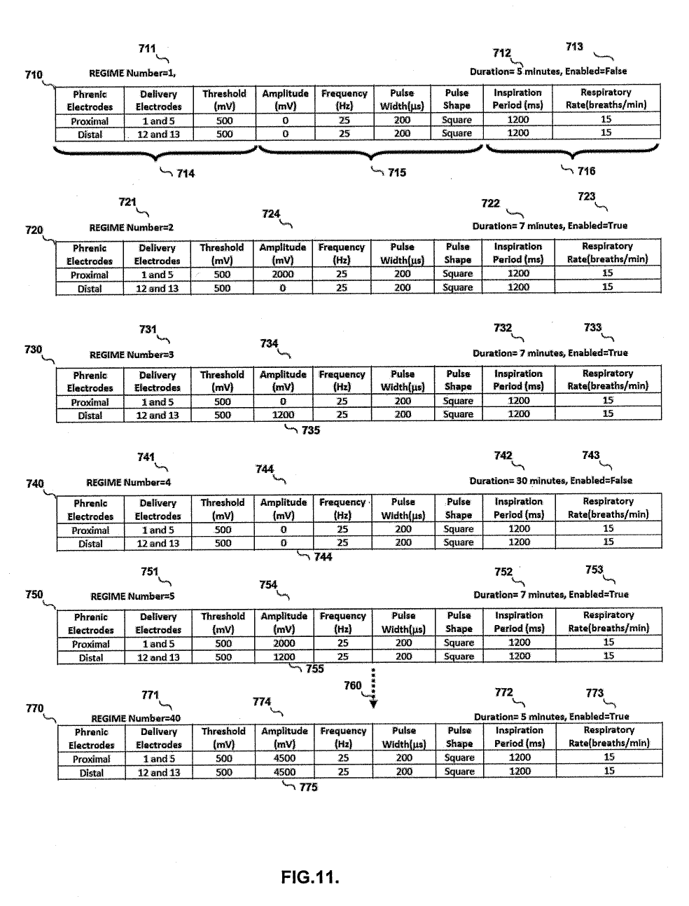

FIG. 11 is an exemplary respiratory support regime list to be used for weaning a patient from mechanical ventilator according to one embodiment. In this example, there is given a total of 40 respiratory support regimes and of these regimes only the regimes 1, 2, 3, 4, 5 and 40 are shown in blocks 710, 720, 730, 740, 750 and 770, respectively. The regimes 6 through 39 are not shown in FIG. 11. In each regime in the list several regime fields are considered. A regime number field 711, a regime duration field 712, a Boolean function field 713 to indicate if the regime is enabled or not, a block of fields 714 containing properties indicating the applicable proximal and distal electrodes (fields include corresponding electrode numbers and their thresholds), a block of fields 715 indicating the parameters of stimulation pulses (parameters include amplitude, frequency, pulse width and pulse shape) and a block fields 716 indicating the details of the respiration therapy (properties include the inspiration period and the respiratory rate) are given. In the exemplary embodiment given in FIG. 11, the regime of block 710 indicates that the proximal electrodes would be 1 and 5 and the distal electrodes would be 12 and 13. The regime number field 711 in FIG. 11, contains a value of 1 indicating that it will be the first regime executed using the flow chart 600 of a method for weaning patients from mechanical ventilators given in FIG. 10. Accordingly, the properties of the proximal and distal electrodes 714 in the regime fields 710 will be activated at a level corresponding to stimulation parameters 715 and respiration therapy properties 716. In the specific example of block 710, the regime number 1 is disabled. However, if it was enabled the proximal electrodes of 1 and 5 would have received square pulses of 500 mV amplitude (500 mV being the threshold voltage) at 200 microsecond duration and 25 Hz repetition frequency. The stimulation would have lasted 1200 ms and then a blanking period of 2800 ms would have applied for expiration to occur to yield a respiration rate of 15 breaths per minute. Similar process would have occurred for the distal electrode pair since both entries for the proximal and distal electrodes are identical in regime block 710. Since this regime is disabled the flowchart of 600 would have branched into block 606 and continued until the duration of 5 minutes specified in duration field of the regime block 712 has expired. Thus the processor would have selected the proximal and distal electrodes but had an activation level of zero.

Regime block 720 has a regime number 2 and therefore would be the next regime that would be selected at block 608 of FIG. 10. Field 723 indicates that this regime is enabled thus the electrode pairs 1 and 5 will be used as proximal and 12 and 13 would be used as the distal phrenic electrodes. The duration field 722 of this regime indicates a value of 7 minutes thus once enabled both proximal and distal electrodes will be activated for 7 minutes. Once activated the proximal electrode pairs 1 and 5 will receive a series of square pulses of 200 microsecond duration at 25 Hz repetition rate and the amplitude of 2500 mV. Of this amplitude value of 2500 mV, the electrode specific threshold of 500 mV is added to the actual therapeutic value of 2000 mV. The distal electrode pairs 12 and 13, however, would receive only 500 mV since the therapeutic value of the stimulation is zero. Thus the patient will receive 2500 mV pulses on the proximal electrodes and 500 mV pulses on the distal electrodes to generate an inspiration of 1200 ms duration in the proximal electrodes and no inspiration on the distal electrodes because the level of stimulation pulses is residing just at the threshold level. Hence the diaphragmatic muscle corresponding to proximal electrodes will be exercised for 7 minutes and the diaphragmatic muscle corresponding to distal electrodes will be at rest. Resultant behavior would be similar to what is being depicted in 510 FIG. 9, where proximal electrodes are activated the distal electrodes are not.

Regime block 730 has a regime number 3 and therefore would be the next regime that would be selected at block 608 of FIG. 10. Field 733 indicates that this regime is enabled thus the electrode pairs 1 and 5 will be used as proximal and 12 and 13 would be used as the distal phrenic electrodes. The duration field 732 of this regime indicates a value of 7 minutes thus once enabled both proximal and distal electrodes will be activated for 7 minutes. Once activated the proximal electrode pairs 1 and 5 will receive a series of square pulses of 200 microsecond duration at 25 Hz repetition rate and the amplitude of 500 mV. Since the level of stimulation pulses is residing just at the threshold level the proximal electrode pair would not be activated. On the other hand, the distal electrode pairs 12 and 13 will receive a series of square pulses of 200 microsecond duration at 25 Hz repetition rate and the amplitude of 1700 mV. Of this amplitude value of 1700 mV, the electrode specific threshold of 500 mV is added to the actual therapeutic value of 1200 mV. Thus the patient will receive 500 mV pulses on the proximal electrodes and 1700 mV pulses on the distal electrodes to generate an inspiration of 1200 ms duration in the distal electrodes and no inspiration on the proximal electrodes because the level of stimulation pulses on this electrode pair is residing just at the threshold level. Hence the diaphragmatic muscle corresponding to distal electrodes will be exercised for 7 minutes and the diaphragmatic muscle corresponding to proximal electrodes will be at rest. Resultant behavior would be similar to what is being depicted in 515 FIG. 9, where distal electrodes are activated the proximal electrodes are not.

Regime block 740 has a regime number 4 and therefore would be the next regime that would be selected at block 608 of FIG. 10. Field 733 indicates that this regime is not enabled but the duration field 742 of this regime indicates a value of 30 minutes. Thus there will no activation of both electrodes and the diaphragmatic muscle will be resting for 30 minutes. Resultant behavior would be similar to what is being depicted in 511 FIG. 9, where both electrodes are not activated.

Regime block 750 has a regime number 5 and therefore would be the next regime that would be selected at block 608 of FIG. 10. Regime field 753 indicates that this regime is enabled thus the electrode pairs 1 and 5 will be used as proximal and 12 and 13 would be used as the distal phrenic electrodes. The duration field 752 of this regime indicates a value of 7 minutes thus once enabled both proximal and distal electrodes will be activated for 7 minutes. Once activated the proximal electrode pairs 1 and 5 will receive a series of square pulses of 200 microsecond duration at 25 Hz repetition rate and the amplitude of 2500 mV. The distal electrode pairs 12 and 13 will receive a series of square pulses of 200 microsecond duration at 25 Hz repetition rate and the amplitude of 1700 mV. Thus the patient will receive 2500 mV pulses on the proximal electrodes and 1700 mV pulses on the distal electrodes to generate an inspiration of 1200 ms duration in the both electrodes but the contraction of the diaphragmatic muscles controlled by the proximal electrodes would be strongly activated than the distal electrodes. Resultant behavior would be similar to what is being depicted in 520 and 525 of FIG. 9, where both electrodes are activated simultaneously.

In FIG. 11 regime blocks 6 through 39 are not depicted but indicated 760. However, it is concluded that a plurality of regimes with variable parameters could be inserted to support any pattern of activation of the diaphragmatic muscles hence tailoring the weaning so that it can be appropriate for a given patient to reduce the weaning time.

Finally, regime block 770 has a regime number 40 and would be the final regime that would be selected at block 608 of FIG. 10. Regime field 773 indicates that this regime is enabled thus the electrode pairs 1 and 5 will be used as proximal and 12 and 13 would be used as the distal phrenic electrodes. The duration field 772 of this regime indicates a value of 5 minutes thus once enabled both proximal and distal electrodes will be activated for 5 minutes. Once activated the proximal electrode pairs 1 and 5 will receive a series of square pulses of 200 microsecond duration at 25 Hz repetition rate and the amplitude of 5000 mV. The distal electrode pairs 12 and 13 will receive a series of square pulses of 200 microsecond duration at 25 Hz repetition rate and the amplitude of 5000 mV. Thus the patient will receive the maximum activation of 5000 mV pulses on both proximal and distal electrodes to generate an inspiration of 1200 ms duration in the both electrodes. Resultant behavior would be similar to what is being depicted in 560 and 565 of FIG. 9, where both electrodes are activated simultaneously.

Thus, methods and devices for providing respiratory or cardiorespiratory support therapy have been presented in the foregoing description with reference to specific embodiments. It is appreciated that various modifications to the referenced embodiments may be made without departing from the scope of the disclosure as set forth in the following claims.

1. A system for providing respiratory support comprising:
an elongate body including a plurality of paired neurostimulation electrodes thereon, said electrodes configured to deliver energy to an area of tissue proximate a right phrenic nerve, a left phrenic nerve or both;
monitoring means for monitoring a respiration amplitude of a patient; and
a controller configured to enable the transmission of energy from the paired electrodes to the tissue proximate the right or left phrenic nerve or both, said controller adapted to
(i) select a first electrode pair of said plurality of neurostimulation electrodes;
(ii) transmit a signal to said first electrode pair to stimulate said tissue proximate said phrenic nerve; and
(iii) receive a monitoring signal from said monitoring means indicating the monitored respiration amplitude of the patient.

2. The system of claim 1 further comprising (iv) if said monitoring signal is indicative of an affirmative respiration amplitude, continue to transmit a signal to said first electrode pair to stimulate said tissue proximate said phrenic nerve to enable respiratory support.

3. The system of claim 1 further comprising (iv) if said signal is not indicative of an affirmative respiration amplitude, transmit a signal to a third pair of electrodes; receive a monitoring signal from said monitoring means indicative of the monitored respiration amplitude of the patient; if said signal is indicative of an affirmative respiration amplitude, continue to transmit a signal to said third pair of electrodes to stimulate said tissue proximate said phrenic nerve to enable respiratory support; and if said monitoring signal is not indicative of an affirmative respiration amplitude, transmit a signal to another pair of electrodes until an affirmative respiration amplitude is received.

4. The system of claim 1 wherein said elongate body is selected from a catheter having a length of from 16 to 30 cm or from 45 to 65 cm.

5. The system of claim 4 wherein said catheter has a diameter from between 4 French to 14 French.

6. The system of claim 1 wherein said plurality of paired electrodes comprise between 2 and 32 electrodes positioned along a portion said elongate body in a spaced-apart relationship.

7. The system of claim 1 wherein said elongate body includes one or more lumens therewithin for receiving a guidewire, one or more injected drugs or saline, or for sampling blood.

8. The system of claim 1 wherein said elongate body further includes an inflatable flow directed balloon adapted to move the catheter and occlude a branch of the pulmonary artery.

9. The system of claim 1 further comprising one or more pressure sensors positioned on said elongate body and adapted to measure venous, cardiac, pulmonary artery and wedge pressures and one or more temperature sensors adapted to measure blood and injected material temperature.

10. The system of claim 1 further comprising a plurality of cardiac pacing and sensing electrodes positioned on said elongate body and adapted to deliver stimulation energy to the heart to pace the chambers of the heart and to measure electrocardiogram.

11. The system of claim 1 wherein the signal is selected from a current amplitude in the range of about 1 to about 20 milliampere; a voltage amplitude in the range of about 1 volts to about 8 volts; a frequency in the range of about 10 to about 100 Hertz (Hz); a pulse width in the range of about 20 to about 400 microseconds; a duty cycle in the range of about 300 ms to 2500 ms; and combinations of the foregoing.

12. The system of claim 1 further comprising one or more of a circuit to sense cardiac electrogram; a circuit to measure blood pressure in the hearts chambers and in the vein; a circuit to measure blood temperature; and a circuit to measure electrical impedance between a selected electrode pair of the plurality of electrodes.

13. The system of claim 1 wherein said controller is configured to (i) determine a start condition for selecting said pair of electrodes; (ii) direct electrical stimulation waveforms to said selected electrodes; and (iii) determine a stop condition to deactivate the selected electrodes.

14. The system of claim 13 wherein said start condition for selection of the electrodes is selected from time measured by a clock; a user input; detection of cardiac or respiratory activity; or a combination of the any of the foregoing.

15. The system of claim 13 wherein said direct electrical stimulation waveforms to said selected electrodes includes selection of proximal pairs of electrodes corresponding to capture of the left phrenic nerve; selection of distal pairs of electrodes corresponding to capture of right phrenic nerve; and selection of proximal and distal pairs of electrodes corresponding to capture of left phrenic nerve and right phrenic nerve.

16. The system of claim 13 wherein said determine a stop condition to deactivate the selected electrodes includes time measured by a clock; a user input; detection of cardiac or respiratory activity; or a combination of the any of the foregoing.

17. The system of claim 16 wherein the detection of respiratory activity includes a change in the electrical impedance between a selected electrode pair of said plurality of electrodes corresponding to respiratory activity; a change in the pressure corresponding to respiratory activity; or a change in the temperature corresponding to respiratory activity.

18. The system of claim 16 wherein the detection of cardiac activity includes a change in the electrical impedance between a selected electrode pair of the plurality of electrodes corresponding to cardiac activity; a change in the blood pressure corresponding to cardiac activity; or a change in the temperature corresponding to cardiac activity.

19. The system of claim 1 further comprising a cardiac signal sensing circuit, wherein said controller is configured to determine whether a cardiac signal is sensed by the cardiac signal sensing circuit by a most distal cardiac sensor positioned in a first position and if said cardiac signal is sensed enabling stimulation of the nerve using a selection of a first bipolar electrode pair in the first position.

20. The system of claim 19 wherein the controller is further configured to select a second bipolar pair of electrodes from the plurality of electrodes in response to sensing a cardiac signal.

21. The system of claim 20 wherein the second bipolar pair of electrodes is configured to stimulate a second nerve.

22. The system of claim 10 wherein the stimulation energy is selected from a pulse width between 0.05 and 5 ms, has an amplitude between 0.5 to 5 volts and has a repetition rate between 40 and 120 beats/minute; and combinations of the foregoing.

23. The system of claim 19 wherein the controller is further configured to schedule nerve stimulation pulses to be delivered using an electrode pair selected from the plurality of electrodes;
determine an electrical impedance between the first bipolar electrode pair of the plurality of electrodes in response to a stimulation of a nerve; and
switch to another electrode pair selected from the plurality of electrodes in response to changes in the electrical impedance to the stimulation of the nerve.

24. A system for providing respiratory support comprising:
a controller;
an elongate body including a plurality of paired neurostimulation electrodes lead connected to the controller;
means for stimulating phrenic nerve tissue;
means for modulating respiration in response to stimulating phrenic nerve stimulation; and
means for dosing the phrenic nerve stimulation.

25. The system of claim 24 wherein said means for dosing is configured to provide dosing on a periodic basis, upon user activation, upon user command, or in response to programmed parameters.

26. The system of claim 24 wherein the programmed parameters comprise stimulation energy.

27. The system of claim 25 wherein the programmed parameters comprise electrode selection.

28. The system of claim 25 wherein the programmed parameters comprise time measured by a clock.
